(19) United States
(12) Patent Application Publication
(10) Pub. No.: US 2013/0195868 A1
(43) Pub. Date: Aug. 1, 2013

(54) TREATING SURFACE OF THE EYE DISORDERS

(71) Applicant: Burt A. Adelman, Cambridge, MA (US)

(72) Inventor: Burt A. Adelman, Cambridge, MA (US)

(73) Assignee: ELEVEN BIOTHERAPEUTICS, INC., Cambridge, MA (US)

(21) Appl. No.: 13/805,175

(22) PCT Filed: Jun. 23, 2011
(86) PCT No.: PCT/US2011/041588

§ 371 Date: Apr. 12, 2013

Related U.S. Application Data
(60) Provisional application No. 61/358,265, filed on Jun. 24, 2010.
(60) Provisional application No. 61/358,248, filed on Jun. 24, 2010.
(60) Provisional application No. 61/360,780, filed on Jul. 1, 2010.
(60) Provisional application No. 61/360,784, filed on Jul. 1, 2010.

(51) Int. Cl. A61K 39/395; A61K 38/17; A61K 38/10; A61K 38/08; A61K 31/56; A61K 31/7105

(52) U.S. Cl. CPC A61K 39/3955; A61K 31/56; A61K 31/7105; A61K 38/10; A61K 38/08; A61K 38/1793
USPC 424/135.1; 424/158.1; 514/44 A; 514/20.8; 424/172.1; 128/887

(57) ABSTRACT

Disclosed herein are methods for administering an IL-1 or IL-17 antagonist for treating ocular surface disorders, e.g., a dry eye disorder. The antagonists can be administered topically using an opthalmic composition.

### CROSS-REFERENCE TO RELATED CASES

This application claims priority to United States provisional applications, U.S. Ser. No. 61/358,248, filed Jun. 24, 2010, U.S. Ser. No. 61/358,265, filed Jun. 24, 2010, U.S. Ser. No. 61/360,780, filed Jul. 1, 2010, and U.S. Ser. No. 61/360,784, filed Jul. 1, 2010, the contents of each of which are hereby incorporated by reference in their entireties.

### BACKGROUND

Ocular surface diseases are disorders affecting the surface of the eye and frequently cause discomfort, visual disturbance, and ocular surface damage. These disorders include conditions also referred to dry eye disease, keratoconjunctivitis sicca, keratitis sicca, sicca syndrome, xerophthalmia, tear film disorder, decreased tear production, aqueous tear deficiency, dysfunctional tear syndrome, and Meibomian gland dysfunction. Two significant mechanisms that can lead to dry eye diseases include: tear hyperosmolarity (which damages ocular surface and activates inflammation) and tear film instability (which results in increased evaporation). Inflammation, both acute and chronic, contributes to dry eye disease progression.

### DESCRIPTION

Disclosed herein are methods of administering an IL-1 and/or IL-17 antagonist to the eye. In one aspect, this disclosure features a method that includes topically administering an ophthalmic composition containing an IL-1 and/or IL-17 antagonist to the eye of a subject prior to sleep or nocturnal rest, e.g., less than one hour, 30 minutes, 20 minutes, 15 minutes, 10 minutes, or 5 minutes or less prior to sleep or nocturnal rest.

The composition can contain any IL-1 and/or IL-17 antagonist and optionally other active ingredients. For example, the IL-1 antagonist is an inhibitory cytokine (e.g., IL-1 Ra or anakinra), an antibody to an IL-1 cytokine (e.g., an antibody that binds and inhibits IL-1α0 or IL-1β), an antibody to an IL-1 receptor, an antibody to an IL-1 receptor accessory protein or other IL-1 antagonist. The composition can be administered to a subject having a disorder described herein, e.g., a chronic inflammatory disorder such as a dry eye syndrome.

In some embodiments, the IL-17 antagonist is an antibody that binds to an IL-17 family member such as IL-17A, IL-17F, IL-17B, IL-17C, IL-17D, and IL-17E, or an antibody to a receptor for an IL-17 family member, e.g., IL-17RA or IL-17RC. In some embodiments, the IL-17 antagonist includes a mutated IL-17 cytokine, e.g., an IL-17 antagonist described in WO 2011/044563. The composition can be administered to a subject having a disorder described herein, e.g., a chronic inflammatory disorder such as a dry eye syndrome.

The ophthalmic composition can be in any of a variety of forms, e.g., an eye drop, dissolvable implant, a filter paper, a gel, an ointment, or a salve. In one embodiment, the ophthalmic composition comprises a matrix, e.g., a biodegradable matrix. The matrix may dissolve or otherwise dissemble subsequent to administration, e.g., after at least about 3, 6, or 9 hours. The matrix may dissolve over an extended period of time, e.g., over the period of at least 3, 6, 9, 12, or 18 hours. In some embodiments, the ophthalmic composition is a gel, e.g., a carbopol gel. The gel can be in the form of a gel strip. In some embodiments, it is a paper or a cotton pledget. In some embodiments, the composition is delivered by a device such as a contact lens, e.g., a soft contact lens.

The composition can be administered to one or both eyes of the subject. The composition can be delivered so that it contacts the cornea and/or the conjunctiva. Typically the composition is self-administered, but can also be administered by a caregiver.

In some embodiments, the ophthalmic composition is administered at least twice daily, and the final dose of the day is administered prior to sleep. For example, the final dose of the day contains a higher concentration of the IL-1 or IL-17 antagonist than earlier doses.

In some embodiments, the method further includes administering an IL-1 or IL-17 antagonist by a parenteral route, e.g., in addition to topical administration. The IL-1 or IL-17 antagonist that is administered parenterally can be the same or different from the IL-1 or IL-17 antagonist in the ophthalmic composition.

The method can include other features described herein.

Also featured herein is a method of administering an IL-1 and/or IL-17 antagonist. The method includes topically administering an ophthalmic composition containing an IL-1 and/or IL-17 antagonist to the eye and subsequently closing the eye for an extended period of time, e.g., for at least 20, 30, 60, 100, 120, 180, 240, or 300 minutes. The method can also include reducing tear drainage. For example, tear drainage is reduced by application of pressure or by occlusion. The method can include other features described herein.

Also featured is a method of administering an IL-1 and/or IL-17 antagonist by topically administering an ophthalmic composition containing an IL-1 and/or IL-17 antagonist to the eye of a subject after an evening meal. The composition can be administered regularly and can be administered at least once each day in the evening or late evening. The method can include other features described herein.

The disclosure also features a method of administering an IL-1 and/or IL-17 antagonist that includes reducing tear drainage, e.g., before, during, or immediately after administering an IL-1 and/or IL-17 antagonist. For example, tear drainage can be reduced by application of pressure or by occlusion. For example, the subject can have a punctal plug inserted into the upper and/or lower punctum of one or both eyes. The plug can include, e.g., collagen implants, silicone, or a hydrogel. The method can include other features described herein.

An IL-1 and/or IL-17 antagonist can be administered, e.g., a subject in a reclining position or who adopts a reclining position, e.g., within one to five minutes of administration. The subject can maintain the reclining position, e.g., a supine position, e.g., for at least at least 20, 30, 60, 100, 120, 180, 240, or 300 minutes. Likewise, the compositions described herein can be provided to the subject with instructions to administer the composition, less than one hour, 30 minutes, 20 minutes, 15 minutes, 10 minutes, or 5 minutes or less prior to bedtime.

In another aspect, this disclosure features a method of administering an IL-1 and/or IL-17 antagonist that includes topically administering an IL-1 and/or IL-17 antagonist and a corticosteroid to the eye of a subject. The IL-1 and/or IL-17 antagonist and the corticosteroid can be administered as a single ophthalmic formulation or as separate formulations. Exemplary corticosteroids include prednisolone, rimexolone, and loteprednol.

The IL-1 and/or IL-17 antagonist and the corticosteroid can be administered to the subject during an induction period, and then the IL-1 and/or IL-17 antagonist can be administered to the subject in the absence of the corticosteroid during a maintenance period. The combination can be used to induce therapy and then the IL-1 and/or IL-17 antagonist alone can be used for maintenance. In some embodiments, the dose of corticosteroid during induction can be less than 1%, 0.5%, or 0.1%. For example, the induction period is less than three months, e.g., less than six, five, three, two, or one weeks. The amount of corticosteroid given can be constant during the induction period or can be varied (e.g., decreased over time). For example, the subject is not given any corticosteroids subsequent to the induction period. The method can include other features described herein.

Also featured are ophthalmic compositions that contain an IL-1 and/or IL-17 antagonist, e.g., as described herein. The ophthalmic compositions described herein can be packaged with instructions, e.g., directing administration according to a method described herein. The instructions can be in a format suitable to guide a patient to self-administer the composition. For example, the instructions can include directions to administer the composition prior to sleep or on a schedule, e.g., wherein at least one dose is at night or prior to closing the eye for an extended time.

The package can include a plurality of compositions, e.g., in single dose form. Some doses can be in higher concentration than others. For example, a higher dosage can be used for nocturnal administration. In some embodiments, some doses can include a combination with a corticosteroid, e.g., as described herein, whereas other doses are not.

Considerable benefits may be realized by administering an IL-1 and/or IL-17 antagonist according to the methods described herein. For example, drainage and clearance of tears is reduced, providing additional time for the IL-1 and/or IL-17 antagonist to reach its target. With respect to disorders affecting the cornea, the IL-1 and/or IL-17 antagonist can bathe or contact the corneal epithelium and reach its targets in the cornea.

The IL-1 and/or IL-17 antagonists described herein can be formulated for ophthalmic delivery, e.g., as an ophthalmic composition. Examples of such compositions include: eye drops, gels, implants, ointments, and other compositions suitable for topical application to the eye or an area in the vicinity of the eye. The compositions can be used to treat inflammation and/or an autoimmune disorder associated with the eye.

### IL-1 Antagonists

Any IL-1 antagonist can be used. Exemplary IL-1 antagonists include antagonists of IL-1β and/or IL-1α, for example:

1. Proteins that bind to IL-1β and, e.g., antagonize IL-1β activity. For example such proteins can prevent interaction between IL-1β and its receptors, e.g., IL-1 R1. Proteins in this category include soluble forms of IL-1β receptors and antibodies that bind IL-1β. Exemplary antibodies that bind to IL-1β include XOMA-052 and canakinumab.

An exemplary antibody can include the following exemplary CDRs:

Light chain:
(SEQ ID NO: 1)
RASQDISNYLS (CDR1),
(SEQ ID NO: 2)
YTSKLHS (CDR2),
(SEQ ID NO: 3)
LQGKMLPWT (CDR3)
Heavy chain:
(SEQ ID NO: 4)
TSGMGVG (CDR1),
(SEQ ID NO: 5)
HIWWDGDESYNPSLK (CDR2),
(SEQ ID NO: 6)
NRYDPPWFVD (CDR3)

Another exemplary antibody includes the following heavy chain variable domain sequence:

(SEQ ID NO: 7)
MEFGLSWVFLVALLRGVQCQVQLVESGGGVVQPGRSLRLSCAASGFTFSVYGMNWVRQAPGKGLE
WVAIIWYDGDNQYYADSVKGRFTISRDNSKNTLYLQMNGLRAEDTAVYYCARDLRTGPFDYWGQG
TLVTVSS

And the following light chain variable domain sequence:

(SEQ ID NO: 8)
MLPSQLIGFLLLWVPASRGEIVLTQSPDFQSVTPKEKVTITCRASQSIGSSLHWYQQKPDQSPKL
LIKYASQSFSGVPSRFSGSGSGTDFTLTINSLEAEDAAAYYCHQSSSLPFTFGPGTKVDI

Proteins that bind to IL-1α and, e.g., antagonize IL-1α activity. Such proteins can prevent interaction between IL-1α and its receptors, e.g., IL-1R1. Proteins in this category include soluble forms of IL-1α receptors and antibodies that bind IL-1α. Still other antagonists are bispecific antibodies that recognize IL-1α and IL-1β. See, e.g., U.S. Pat. No. 7,612,181.

3. Receptor-derived proteins that bind to one or both of IL-1β and IL-1α. Examples include soluble forms of receptors for IL-1 cytokines, e.g., soluble forms of IL-1R1 and IL-1 R2, and IL-1RAcP. An example of such a protein is rilonacept.

4. Proteins that bind to IL-1R1 or otherwise antagonize IL-1R1. Examples of such proteins include IL-1Ra and related proteins. Still other examples include antibodies that bind to IL-1R1 and peptides that bind to IL-1 R1.

An exemplary protein that includes IL-1Ra is methionyl IL-1Ra and anakinra (marketed as Kineret, Amgen, Thousand Oaks Calif., USA). An exemplary amino acid sequence for IL-1Ra includes:

(SEQ ID NO: 9)
RPSGRKSSKM QAFRIWDVNQ KTFYLRNNQL VAGYLQGPNV NLEEKIDVVP IEPHALFLGI
HGGKMCLSCV KSGDETRLQL EAVNITDLSE NRKQDKRFAF IRSDSGPTTS FESAACPGWF
LCTAMEADQP VSLTNMPDEG VMVTKFYFQE DE

The protein can be a methionyl form of human IL-1Ra and variants, e.g., including those known in the art such as in U.S. Pat. No. 5,922,573, Evans et al. (1995), J. Biol. Chem. 270: 11477-11483; Greenfeder, et al. (1995) J. Biol. Chem. 270: 22460-22466, and Boraschi, et al. (1996) Frontiers in Bioscience1:d270-308 (PubMed ID 9159234).

Additional exemplary IL-1 antagonists include peptides that bind to and inhibit IL-1R1. Peptides can bind, e.g., with an affinity of less than 500 nM, 50 nM, or 1 nM. Peptides can include between 8 and 40 amino acids, e.g., between 10-30 amino acids. Exemplary peptides that bind to inhibit IL-1R1 include peptides described in U.S. Pat. No. 5,861,476. Exemplary peptides can include the following amino acid sequences: FTWEESNAYYWQPY (SEQ ID NO: 10); ETPFTWEESNAYYWQPYALPL (SEQ ID NO: 11); FEWTPGYWQPY-NH2 (SEQ ID NO: 12); FEWTPGYWQHY-NH2 (SEQ ID NO: 13); FEWTPGWYQJY-NH2 (SEQ ID NO: 14); AcFEWTPGWYQJY-NH2 (SEQ ID NO: 15); FEWTPGW-pY-QJY-NH2 (SEQ ID NO: 16); FAWTPGYWQJY-NH2 (SEQ ID NO: 17); FEWAPGYWQJY-NH2 (SEQ ID NO: 18); where AcF is acetylated phenylalanine, J is azetidine, Y-NH2 is tyrosinamide, and -pY- is phosphotyrosine. Methods for synthesizing peptides are described, e.g., in Peptide Synthesis and Applications (Howl, ed.), Humana Press (2010) (ISBN: 1617374903). Still other exemplary peptides include the amino acid sequences described in U.S. Pat. No. 5,861,476.

5. Proteins that bind to IL-1 RAcP or otherwise antagonize IL-1RAcP. Such antagonists include antibodies that bind to IL-1RAcP. For example such antibodies can prevent IL-1RAcP interaction with IL-1R1. Antibodies can be generated, e.g., by immunization and/or display technologies.

6. Nucleic acid antagonists Nucleic acid antagonists can be used to decrease IL-1 activity, e.g., siRNA and other interfering RNAs can be used to antagonize mRNA encoding components of the IL-1 signaling pathway. Antagonistic RNAs can be identified based on the known sequences for components of the IL-1 signaling pathway. Exemplary antagonist RNAs sequences can have include the RNAs that comprise one of the following sequences: UGUAAACAUCCUACACUCUCAGC (SEQ ID NO: 19); UGUAAACAUCCUACACUCAGC (SEQ ID NO: 20); UGUAAACAUCCUCGACUGGAAGC (SEQ ID NO: 21); UGGCUCAGUUCAGCAGGAACAG (SEQ ID NO: 22); UAUGGCUUUUCAUUCCUAUAGUG (SEQ ID NO: 23); CCUCUGGGCCCUUCCUCCAG (SEQ ID NO: 24); UGGACGGAGAACUGAUAAGGGU (SEQ ID NO: 25); ACAGCAGGCACAGACAGGCAG (SEQ ID NO: 26); GUGAAAUGUUUAGGACCACUAG (SEQ ID NO: 27); GCCCCUGGGCCUAUCCUAGAA (SEQ ID NO: 28); UCCUUCAUUCCACCGGAGUCUG (SEQ ID NO: 29).

Still other exemplary antagonists include small molecules that decrease IL-1β and/or IL-1α activity.

### IL-17 Antagonists

Any IL-17 antagonist can be used. As described herein, the term “IL-17 antagonist” encompasses antagonists of all IL-17 family members, e.g., antagonists of IL-17A, IL-17F, IL-17B, IL-17C, IL-17D, and IL-17E, particularly the human forms of these proteins.

Exemplary IL-17 antagonists include:
agents (such as antibodies and other binding proteins) that bind to IL-17 family members (including IL-17A, IL-17F, IL-17B, IL-17C, IL-17D, and IL-17E) and which antagonize IL-17 mediated signaling;
agents (such as antibodies and other binding proteins including proteins described in WO 2011/044563) that bind to one or more receptors for IL-17, such as IL-17RA and IL-17RC and which antagonize signaling mediated by IL-17 family members;
agents (such as antibodies and other binding proteins) that bind to a complex containing IL-17 and at least one receptor subunit, e.g., IL-17 and II-17RA, or IL-17, IL-17RA, and IL-17RC and which antagonize signaling mediated by IL-17 family members; and
agents such as soluble receptors that include one or more of soluble extracellular domains of IL-17RA and IL-17RC and which antagonize signaling mediated by IL-17 family members.

Exemplary antibodies to IL-17A can bind with an equilibrium dissociation constant (K_{D}) of less than 10⁻⁷, 10⁻⁸, 10⁻⁹, or 10⁻¹⁰ M. Exemplary antibodies to IL-17A include antibodies having one or more of the following immunoglobulin variable domains or sequences at least 80, 85, 90, or 95% identical to such sequences:

Heavy chain:
(SEQ ID NO: 30)
EVQLVESGGGLVQPGGSLRLSCAASGFTFSNYWMNWVRQAPGKGLEWVAAINQDGSEKYYVGSVK
GRFTISRDNAKNSLYLQMNSLRVEDTAVYYCVRDYYDILTDYYIHYWYFDLWGRGTLVTVSS
Light chain:
(SEQ ID NO: 31)
EIVLTQSPGTLSLSPGERATLSCRASQSVSSSYLAWYQQKPGQAPRLLIYGASSRATGIPDRFSG
SGSGTDFTLTISRLEPEDFAVYYCQQYGSSPCTFGQGTRLEIKR

Another exemplary antibody can include: one or more of the following immunoglobulin variable domains or sequences at least 80, 85, 90, or 95% identical to such sequences:

Heavy chain:
(SEQ ID NO: 32)
EVQLLESGGGLVQPGGSLRLSCAASGFTFSSYAMSWVRQAPGKGLEWVSAISGSGGSTYYADSVK
GRFTISRDNSKNTLYLQMNSLRAEDTAVYYCARDLIHGVTRNWGQGTLVTVSS
Light chain:
(SEQ ID NO: 33)
NFMLTQPHSVSESPGKTVTISCTRSSGSLANYYVQWYQQRPGSSPTIVIFANNQRPSGVPDRFSG
SIDSSSNSASLTISGLKTEDEADYYCQTYDPYSVVFGGGTKLTVLGE

Still another exemplary antibody is LY2439821. See Genovese, Arthritis Rheum. 2010 Apr;62(4):929-39.

Exemplary antibodies to IL-17 can bind with an equilibrium dissociation constant (K_{D}) of less than 10⁻⁷, 10⁻⁸, 10⁻⁹, or 10⁻¹⁰ M. Still other exemplary antibodies are antibodies that bind to both IL-17A and IL-17F with an equilibrium dissociation constant (K_{D}) of less than 10⁻⁷, 10⁻⁸, 10⁻⁹, or 10⁻¹⁰ M.

In one embodiment, the IL-17 antagonist is an agent that binds to a receptor for IL-17, such as IL-17RA and IL-17RC, and prevents the receptor from engaging with a second IL-17 receptor. For example, the antagonist binds to IL-17RA and prevents IL-17RA from engaging IL-17RC, or the antagonist binds to IL-17RC and prevents IL-17RC from engaging IL-17RA. Agents that bind to receptors (such as antibodies) can bind with an equilibrium dissociation constant (K_{D}) of less than 10⁻⁷, 10⁻⁸, 10⁻⁹, or 10⁻¹⁰ M. Exemplary antagonists are also described in WO 2011/044563.

### Ocular Disorders and Delivery

IL-1 and/or IL-17 antagonists and methods disclosed herein can be used to treat ocular disorders, including ocular disorders affecting the surface of the eye, ocular disorders mediated at least in part by an immune reaction, and ocular disorders associated with an IL-1 mediated autoimmune condition (such as Sjögren's disease and rheumatoid arthritis). The patient may or may not have other manifestations of a systemic autoimmune disorder. The antagonist is administered according to a method described herein.

The ocular disorder can be a dry eye disorder that affects the surface of the eye. The disorder includes conditions also referred to keratoconjunctivitis sicca, keratitis sicca, sicca syndrome, xerophthalmia, tear film disorder, decreased tear production, aqueous tear deficiency, and Meibomian gland dysfunction. Dry eye can include forms that are associated with Sjögren syndrome, e.g., Sjögren syndrome associated keratoconjunctivitis sicca, but also forms that are not so associated, e.g., non-Sjögren syndrome associated keratoconjunctivitis sicca. The patient may or may not have other manifestations of a systemic autoimmune disorder.

Subjects having a dry eye syndrome can exhibit inflammation of the eye, and can experience scratchy, stingy, itchy, burning or pressured sensations, irritation, pain, and redness. Dry eye can be associated with both excessive eye watering and conversely insufficient tear production. An IL-1 and/or IL-17 antagonist can be administered to such subjects to ameliorate or prevent the onset or worsening of one or more such symptoms.

An IL-1 and/or IL-17 antagonist can also be used to treat other disorders affecting the surface of the eye, such as the cornea. Such disorders include corneal ocular surface inflammatory conditions, corneal neovascularization, keratitis, including peripheral ulcerative keratitis and microbial keratitis. An IL-1 and/or IL-17 antagonist can be used to treat disorders affecting the conjunctiva, including conjunctival scarring disorders and conjunctivitis. The IL-1 and/or IL-17 antagonist can be used to treat still other disorders such as pemphigoid syndrome and Stevens-Johnson syndrome.

An IL-1 and/or IL-17 antagonist can be administered to a subject who is about to receive, undergoing, or recovering from a procedure involving the eye, e.g., corneal transplantation/keratoplasty, keratoprosthesis surgery, lamellar transplantation, selective endothelial transplantation. An IL-1 antagonist described herein can be administered to a subject to modulate neovascularization in or around the eye.

An IL-1 and/or IL-17 antagonist can be administered to a subject having an allergic reaction affecting the eye, e.g., a subject experiencing severe allergic (atopic) eye disease.

An IL-1 and/or IL-17 antagonist can be administered to a subject having an autoimmune disorder affecting the eye. Exemplary autoimmune ocular disorders include sympathetic ophtahlmia, Vogt-Koyanagi Harada (VKH) syndrome, birdshot retinochoriodopathy, ocular cicatricial pemphigoid, Fuchs' heterochronic iridocyclitis, and various forms of uveitis. An IL-1 and/or IL-17 antagonist can be administered to a subject to treat any of the foregoing disorders.

Uveitis includes acute and chronic forms, and includes inflammation of one or more of the iris, the ciliary body, and the choroid. Chronic forms may be associated with systemic autoimmune disease, e.g., Behcet's syndrome, ankylosing spondylitis, juvenile rheumatoid arthritis, Reiter's syndrome, and inflammatory bowel disease. In anterior uveitis, inflammation is primarily in the iris (also iritis). Anterior uveitis can affect subject who have systemic autoimmune disease, but also subjects who do not have systemic autoimmune disease. Intermediate uveitis involves inflammation of the anterior vitreous, peripheral retina, and ciliary body, often with little anterior or chorioretinal inflammation. Pan planitis results from inflammation of the pars planana, between the iris and the choroid. Posterior uveitis involves the uveal tract and primarily the choroid, and is also referred to as choroiditis. Posterior uveitis can be associated with a systemic infection or an autoimmune disease. It can persist for months and even years. An IL-1 antagonist can be administered to a subject to treat any of the foregoing forms of uveitis.

An IL-1 and/or IL-17 antagonist can be administered to a subject having a systemic autoimmune disorder, e.g., rheumatoid arthritis, juvenile rheumatoid arthritis, ankylosing spondylitis, Reiter's syndrome, scleroderma, and an inflammatory bowel disease. The subject can be at risk for or exhibiting an ocular symptom.

An IL-1 and/or IL-17 antagonist described herein is generally delivered directly to the eye or in the vicinity of the eye. For example, the protein can be administered topically or intraocularly, e.g., as described below.

Treating a disorder described herein includes administration of an antagonist described herein to a subject, e.g., a patient, in an amount, manner, and/or mode effective to improve a condition, symptom, or parameter associated with a disorder, or to prevent progression of a disorder, to either a statistically significant degree or to a degree detectable to one skilled in the art. The treatment can be to intended to cure, heal, alleviate, relieve, alter, remedy, ameliorate, palliate, improve or affect the disorder, the symptoms of the disorder or the predisposition toward the disorder. An effective amount, manner, or mode can vary depending on the subject and may be tailored to the subject. Exemplary subjects include humans, primates, and other non-human mammals.

The subject can be administered an antagonist, for example, after being evaluated for one or more symptoms of a disorder described herein. The subject can be free of any bacterial or viral infections in the eye. An IL-1 and/or IL-17 antagonist described herein can be administered to a subject who is a non-responder to treatment with cyclosporine or artificial tears. A non-responder to treatment with a particular agent is a subject who does not show a significant improvement after treatment with the agent. For example, non-responders include subjects who do not show a significant improvement or who show deterioration after at least one week, one, two, three, four, six, or nine months of treatment.

### Formulations and Methods for Ocular Delivery

Ophthalmic compositions can be delivered by a topical administration and can be formulated for administration as a liquid drop or an ointment, or for implantation, e.g., into an anterior chamber of the eye or the conjunctival sac. For example, the formulation can be a solution, a suspension, an emulsion, an ointment, a gel, or a spray. Liquid drops can be delivered using an eye dropper. When formulated for ocular delivery, the IL-1 and/or IL-17 antagonist can be present at 0.01-5%, e.g., 0.1-2%, or 1%-5% concentration. For example, the IL-1 and/or IL-17 antagonist is IL-1Ra and is administered at a concentration of 0.01-5%, e.g., 0.1-2%, or 1%-5%, e.g., between 1-3%.

Frequently the ophthalmic formulation is applied directly to the eye including topical application to the eyelids or instillation into the space (cul-de-sac) between the eyeball and the eyelids. The ophthalmic formulation can be designed to mix readily with the lacrimal fluids and spread over the surfaces of the cornea and conjunctiva. This can deposit the antagonist in the lower fornix. Capillarity, diffusional forces, and the blinking reflex drive incorporation of the antagonist in the precorneal film from which it penetrates into and through the cornea.

Ophthalmic formulations can also include one or more other agents, e.g., an anti-inflammatory steroid or corticosteroid such as prednisolone, rimexolone, loteprednol, medrysone and hydrocortisone, or a non-steroidal anti-inflammatory. For example, the steroid can be present at a concentration of 0.001 to 1%. Exemplary formulations include prednisolone at 0.5-2%, 0.5-1% or about 1%, or rimexolone at 0.5-2%, 0.5-1%, or about 1%, or loteprednol at 0.1-1%, 0.1-0.5%, or about 0.5%. Corticosteroid can also be administered in a separate formulation from the IL-1 antagonist.

In some embodiments, the subject is administered a formulation that contains a low dose of corticosteroid, e.g., less than 0.2, 0.1, or 0.05%. For example, the formulation having the IL-1 antagonist is formulated together with prednisolone at less than 0.2% or 0.01%, 0.05%, or 0.02% or about 0.05%, or rimexolone at 0.2% or 0.01%, 0.05%, or 0.02% or about 0.05%, or loteprednol at 0.1% or 0.05%, 0.02%, or 0.01% or about 0.01%. Low dose corticosteroid can also be administered as a separate formulation.

The formulation can also include one or more of the following components: surfactants, tonicity agents, buffers, preservatives, co-solvents and viscosity building agents. Tonicity agents can be used to adjust the tonicity of the composition, e.g., to that of natural tears. For example, potassium chloride, sodium chloride, magnesium chloride, calcium chloride, dextrose and/or mannitol may be added to achieve an appropriate tonicity, e.g., physiological tonicity. Tonicity agents can be added in an amount sufficient to provide an osmolality of about 150-450 mOsm or 250-350 mOsm.

The formulation can also include buffering suitable for ophthalmic delivery. The buffer can include one or more buffering components (e.g., sodium phosphate, sodium acetate, sodium citrate, sodium borate or boric acid) to changes in pH especially under storage conditions. For example, the buffer can be selected to provide a target pH within the range of pH 6.0-7.5, e.g., 6.5-7.5.

The formulation can include an aqueous or phospholipid carrier. Particularly for treating dry eye disorders, the formulation can include agents to provide short-term relief, e.g., compounds which lubricate the eye and assist in tear formation. For example, phospholipid carriers (which include one or more phospholipids) can be used to provide short-term relief. Examples or artificial tears compositions useful as artificial tears carriers include, but are not limited to, commercial products, such as Tears Naturale™ (Alcon Labs, Inc., Tex. USA). For example, per ml, the formulation can include: 1 mg dextran 70 and 3 mg hydroxypropyl methylcellulose, and optionally a preservative such POLYQUAD® (polyquaternium-1) 0,001% (m/v). Examples of phospholipid carrier formulations include those disclosed in U.S. Pat. Nos. 4,804,539, 4,883,658, 5,075,104, 5,278,151, and 5,578,586.

The formulation can also include other compounds that act as a lubricant or wetting agent. These include viscosity agents such as: monomeric polyols, such as, glycerol, propylene glycol, ethylene glycol; polymeric polyols, such as, polyethylene glycol, various polymers of the cellulose family: hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941; carbomer 940, carbomer 974P. Still additional examples include polysaccharides, such as hyaluronic acid and its salts, chondroitin sulfate and its salts, and acrylic acid polymers. In certain embodiments, the formulation has a viscosity of 1 to 400 cP.

An ophthalmic gel that includes an IL-1 and/or IL-17 antagonist can be formulated, e.g., using a carbopol gel. An exemplary gel includes one or more of hydroxypropyl methylcellulose, sodium perborate, phosphoric acid, and sorbitol. For example, carbopol 980 can be used.

An IL-1 and/or IL-17 antagonist can be formulated in an ophthalmic pack that provides prolonged contact of an ophthalmic formulation with the eye. For example, a cotton pledget is saturated with the formulation and then inserted into the superior or inferior fornix.

An IL-1 and/or IL-17 antagonist can be formulated with hydroxypropyl cellulose as an insert. The hydroxypropyl cellulose can provide a reservoir of the antagonist. The matrix can absorb fluid and soften upon application and can then dissolve, e.g., be substantially dissolved by 12, 14, or 16 hours. The insert can be administered, e.g., to the inferior cul-de-sac. See e.g., U.S. Pat. No. 4,343,787.

An IL-1 and/or IL-17 antagonist can be administered using a filter paper strip soaked with the antagonist. The antagonist can be administered using a corneal shield, e.g., formulated from a collagen matrix.

An IL-1 and/or IL-17 antagonist can be impregnated into a contact lens, particularly a soft contact lens. Soft contact lenses can be made of, for example, acofilcon A, alofilcon A, alphafilcon A, amfilcon A, astifilcon A, atlafilcon A, balafilcon A, bisfilcon A, bufilcon A, comfilcon A, crofilcon A, cyclofilcon A, deltafilcon, dimefilcon A, droxfilcon A, elastofilcon A, epsilficon A, esterifilcon A, etafilcon A, genfilcon A, govafilcon A, hefilcon, hilafilcon, hioxifilcon, hydrofilcon A, lenefilcon A, licryfilcon, lidofilcon, lotrafilcon, mafilcon A, mesafilcon A, methafilcon B, mipafilcon A, nelfilcon A, netrafilcon A, ocufilcon, ofilcon A, omafilcon A, oxyfilcon A, perfilcon A, pevafilcon A, phemfilcon A, polymacon, senofilcon A, silafilcon A, siloxyfilcon A, tefilcon A, tetrafilcon A, trilfilcon A, vifilcon, and xylofilcon A. The subject can wear the soft contact lens at least overnight. For example, the subject can insert the contact lens before sleep (e.g., at night) and wear the contact lens at least overnight during which time, the IL-1 and/or IL-17 antagonist is released and delivered to the eye and ocular tissue, e.g., the cornea.

The formulation can be packaged for single or multi-dose use, e.g., in a bottle with an associated dropper or as a set of single-use droppers. The formulation can include one or more preservatives, e.g., prevent microbial and fungal contamination during use. Exemplary preservatives include: benzalkonium chloride, chlorobutanol, benzododecinium bromide, methyl paraben, propyl paraben, phenylethyl alcohol, purite, edetate disodium, sorbic acid, sodium benzoate, sodium proprionate, sodium perborate, and polyquaternium-1, and can be included at a concentration of from 0.001 to 1.0% w/v. It is also possible to provide formulations containing an IL-1 antagonist that are sterile yet free of preservatives. The formulations can be prepared for single use application.

With respect to dry eye and other surface disorders, subjects can be evaluated using one or more of the following approaches: the Ocular Surface Disease Index

(OSDI), corneal and conjunctival staining, and the Schirmer test. The methods described herein can be used to improve at least one parameter associated with one or more of these approaches.

The Ocular Surface Disease Index (OSDI) is a 12-item questionnaire that provides a rapid assessment of the symptoms of ocular irritation consistent with ocular surface inflammatory disorders, including dry eye disorders, and their impact on vision-related functioning. See e.g. Ocul. Immunol. Inflamm. 2007 Sep-Oct;15(5):389-93. The 12 items of the OSDI questionnaire are graded on a scale of 0 to 4. Scores are derived based on responses to provide an OSDI score on a scale of 0 to 100, with higher scores representing greater disability. A negative change from baseline indicates an improvement in vision-related function and the ocular inflammatory disorders.

Corneal and Conjunctival Staining: Corneal staining is a measure of epithelial disease, or break in the epithelial barrier of the ocular surface, typically seen with ocular surface inflammatory disorders such as dry eye. Corneal staining can exist even without clinically evident dry eye, e.g., as with significant lid disease, such as posterior blepharitis. Corneal staining is highly correlated with ocular discomfort in many, though not all, patients; in general corneal staining is associated with high scores in the OSDI, as described above. For corneal fluorescein staining, saline-moistened fluorescein strips or 1% sodium fluorescein solution are used to stain the tear film. The entire cornea is then examined using slit-lamp evaluation with a yellow barrier filter (#12 Wratten) and cobalt blue illumination. Staining is graded according to the Oxford Schema. Conjunctival staining is likewise a measure of epithelial disease or break in the epithelial barrier of the ocular surface. Conjunctival staining is performed under the slit-lamp using lissamine green. Saline-moistened strip of 1% lissamine green solution is used to stain the tear film, and interpalpebral conjunctival staining is evaluated more than 30 seconds, but less than 2 minutes, later. Using white light of moderate intensity, only the interpalpebral region of the nasal and temporal conjunctival staining is graded using the Oxford Schema. Schirmer Test: The Schirmer test is performed in the presence and in the absence of anesthesia by placing a narrow filter-paper strip (5×3 5 mm strip of Whatman #41 filter paper) in the inferior cul-de-sac. This test is conducted in a dimly lit room. The patient gently closes his/her eyes until five minutes have elapsed and the strips are removed. Because the tear front will continue advancing a few millimeters after it has been removed from the eyes, the tear front is marked with a ball-point pen at precisely five minutes. Aqueous tear production is measured by the length in millimeters that the strip wets during 5 minutes. Results of 10 mm or less for the Schirmer test without anesthesia and 5 mm or less for the Schirmer test with anesthesia are considered abnormal. A positive change from baseline indicates improvement of one or more symptoms of an ocular inflammatory disorder described herein.

In one embodiment, prior to, during, or after administering an IL-1 and/or IL-17 antagonist, the subject is treated to reduce the rate of tear drainage. For example, the subject can be treated to at least partially occlude drainage, e.g., by insertion of a punctal plug, e.g., a plug formed of one or more of a collagen matrix, silicone, a hydrogel, or an acrylic. The plug can be dissolvable or non-dissolvable. Exemplary manufacturers of plugs include Lacrimedics, Eaglevision, Oasis, Angiotech, Odyessy, and FCI Ophthalmics, among others.

Generally, when an IL-1 and/or IL-17 antagonist is administered, the subject has removed any contact lenses prior to administration.

### Antibody Generation:

Exemplary IL-1 and/or IL-17 antagonists are antibodies. As used herein, the term “antibody” refers to a protein that includes at least one immunoglobulin variable region. For example, an antibody can include a heavy chain variable region (VH), and a light chain variable region (VL). In another example, an antibody includes two VH regions and two VL regions. The term “antibody” encompasses antigen-binding fragments of antibodies (e.g., single chain antibodies, Fab fragments, F(ab′)₂ fragments, Fd fragments, Fv fragments, and dAb fragments) as well as complete antibodies, e.g., intact immunoglobulins of types IgA, IgG, IgE, IgD, IgM (as well as subtypes and modified versions thereof). Still other antibodies only include a single immunoglobulin variable domain. See, e.g., Janssens et al. Proc Natl Acad Sci USA, 103(41):15130-5 (2006).

The VH and VL regions can be further subdivided into regions of hypervariability, termed “complementarity determining regions” (“CDR”), interspersed with regions that are more conserved, termed “framework regions” (FR). The extent of the FRs and CDRs has been precisely defined (see, Kabat, E.A., et al. (1991) Sequences of Proteins of Immunological Interest, Fifth Edition, U.S. Department of Health and Human Services, NIH Publication No. 91-3242; and Chothia, C. et al. (1987) J. Mol. Biol. 196:901-917). Kabat definitions are used herein. Each VH and VL is typically composed of three CDRs and four FRs, arranged from amino-terminus to carboxyl-terminus in the following order: FR1, CDR1, FR2, CDR2, FR3, CDR3, FR4. The canonical structures of hypervariable loops of an immunoglobulin variable can be inferred from its sequence, as described in Chothia et al. (1992) J. Mol. Biol. 227:799-817; Tomlinson et al. (1992) J. Mol. Biol. 227:776-798); and Tomlinson et al. (1995) EMBO J. 14(18):4628-38. To generate an antibody, an animal can be immunized with the intended target (e.g., IL-1β, IL-1α, the extracellular domain of IL-1 R1, the extracellular domain of IL-1 RAcP, IL-17A, IL-17F, IL-17B, IL-17C, IL-17D, and IL-17E, the extracellular domain of IL-17RA, or the extracellular domain of IL-17RC) or a fragment of the target and used as a source of antibodies or antibody sequences (e.g., CDR sequences). The antibodies can be produced using standard hybridoma technology. It is also possible to engineer mouse strains deficient in mouse antibody production with human Ig sequences. Antigen-specific monoclonal antibodies derived from the genes encoding antibodies with the desired specificity may be produced and selected. See also, e.g., XenoMouse™, Green et al. Nature Genetics 7:13-21 (1994), U.S. 2003-0070185, U.S. Pat. No. 5,789,650, and WO 96/34096. Non-human antibodies to the target can also be produced, e.g., in a rodent. The non-human antibody can be humanized, e.g., as described in U.S. Pat. No. 6,602,503, EP239400, U.S. Pat. No. 5,693,761, and U.S. Pat. No. 6,407,213. Antibodies with the desired properties can be identified by screening the antibodies in any of the assays described herein. For example, antibodies can be identified for ability to inhibit IL-1β or IL-1α activity. All or a fragment of the generated antibody, e.g., a Fab, Fab′, F(ab′)₂ or Fv fragment, can be cloned and used as an IL-1 antagonist. For example, antibodies can be identified for ability to inhibit signaling mediated by IL-17 family members. All or a fragment of the generated antibody, e.g., a Fab, Fab′, F(ab′)₂ or Fv fragment, can be cloned and used as an IL-17 antagonist.

EP 239 400 (Winter et al.) describes altering antibodies by grafting (within a given variable region) complementarity determining regions (CDRs) from the antibody of one species to those from another. See also Riechmann et al., 1988, Nature 332, 323-327; Verhoeyen et al., 1988, Science 239, 1534-1536). Typically, CDRs of a murine antibody are grafted into the corresponding regions in a human antibody by using recombinant nucleic acid technology to produce sequences encoding the desired substituted antibody. Human constant region gene segments of the desired isotype (usually gamma 1 for CH and kappa for CL) can be added and the humanized heavy and light chain genes can be co-expressed in mammalian cells to produce soluble humanized antibody. A fragment of the humanized antibody is suitable for use in a binding agent.

Queen et al., 1989 and WO 90/07861 describe a process that includes choosing human V framework regions by computer analysis for optimal protein sequence homology to the V region framework of the original murine antibody and modeling the tertiary structure of the murine V region to visualize framework amino acid residues that are likely to interact with the murine CDRs. These murine amino acid residues are then superimposed on the homologous human framework. See also U.S. Pat. Nos. 5,693,762; 5,693,761; 5,585,089; and U.S. Pat. No. 5,530,101.

The antibodies described herein can be provided in any of a variety of formats, including as immunoconjugates, intrabodies, and multivalent antibodies, e.g., bivalent antibodies, triabodies, tetravalent antibodies, peptabodies and hexabodies. Still other configurations include “kappabodies” (III et al. Protein Eng 10:949-57 (1997)), “minibodies” (Martin et al. EMBO J13:5303-9 (1994)), “diabodies” (Holliger et al. Proc Natl Acad Sci USA 90:6444-6448 (1993)), and “Janusins” (Traunecker et al. EMBO J 10:3655-3659 (1991) and Traunecker et al. Int J Cancer Suppl 7:51-52 (1992)). Furthermore, the effector function of an antibody may be changed by isotype switching to an IgG1, IgG2, IgG3, IgG4, IgD, IgA1, IgA2, IgE, or IgM for various therapeutic uses. Constant domains that lack effector function can also be used. Antibodies may also include modifications, e.g., modifications that alter Fc function, e.g., to decrease or remove interaction with an Fc receptor or with C1q, or both. For example, the human IgG1 constant region can be mutated at one or more residues, e.g., one or more of residues corresponding to residues 294 and 297, to alter Fc function (Arnold et al., Ann. Rev. Immunol. 25: 21-50 (2007)).

In addition to antibodies, other binding agents can be used as antagonists. Such agents can be identified using display technology. A display library is a collection of entities, where each entity includes an accessible polypeptide component and a recoverable component that encodes or identifies the polypeptide component. Typically the recoverable component is a cell (such as yeast) or virus (such as a bacteriophage). A variety of formats can be used for display libraries, such as cell/yeast display (e.g., Chao et al. Nat Protoc. 2006;1(2):755-68; Colby et al. Methods Enzymol. 2004;388:348-58; Boder et al., Methods Enzymol. 2000;328:430-44), phage display (e.g., Viti et al., Methods Enzymol. 2000;326:480-505 and Smith (1985) Science 228:1315-1317), ribosome display (e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30; and Schaffitzel et al. (1999) J Immunol Methods. 231(1-2):119-3

The polypeptide component of the display library is varied so that different amino acid sequences are represented. The polypeptide component can be of any length and can include more than one polypeptide component, for example, the two polypeptide chains of a Fab. In one exemplary implementation, a display library can be used to identify proteins that bind to the intended target, e.g., IL-1β, IL-1α, the extracellular domain of IL-1 R1, the extracellular domain of IL-1 RAcP, IL-17A, IL-17F, IL-17B, IL-17C, IL-17D, and IL-17E, the extracellular domain of IL-17RA, or the extracellular domain of IL-17RC. In a selection, the polypeptide component of each member of the library is probed with the target (or fragment thereof) and if the polypeptide component binds to the target, the display library member is identified, typically by retention on a support.

A wide variety of scaffolds are available. See, e.g., Hosse et al., Protein Science, 15:14-27 (2006). Examples of scaffolds for a binding moiety include: antibodies (e.g., Fab fragments, single chain Fv molecules (scFV), single domain antibodies, camelid antibodies, and camelized antibodies); T-cell receptors; MHC proteins; extracellular domains (e.g., fibronectin Type III repeats, EGF repeats); protease inhibitors; TPR repeats; trifoil structures; enzymes, e.g., proteases (particularly inactivated proteases), chaperones, e.g., thioredoxin and heat shock proteins; intracellular signaling domains (such as PDZ, SH2 and SH3 domains); linear and constrained peptides; and linear peptide substrates.

Antagonists described herein also include compounds that are at least 70, 75, 77, 80, 82, 85, 87, 88, 89, 90, 91, 92, 93, 94, 95, 96, 97, 98, or 99% identical to sequences disclosed herein. Antibodies can include CDR sequences that are at least 70, 75, 77, 80, 82, 85, 87, 88, 89, 90, 91, 92, 93, 94, 95, 96, 97, 98, or 99% identical to CDR sequences of an antibody disclosed herein and which bind to the target of such antibody.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

### Production

IL-1 antagonists and IL-17 antagonists can be produced by expression in recombinant host cells, but also by other methods such as in vitro transcription and translation and chemical synthesis.

One or more nucleic acids (e.g., cDNA or genomic DNA) encoding an IL-1 antagonist or IL-17 antagonist may be inserted into a replicable vector for cloning or for expression. Various vectors are publicly available. The vector may, for example, be a plasmid, cosmid, viral genome, phagemid, phage genome, or other autonomously replicating sequence. The appropriate coding nucleic acid sequence may be inserted into the vector by a variety of procedures. For example, appropriate restriction endonuclease sites can be engineered (e.g., using PCR). Then restriction digestion and ligation can be used to insert the coding nucleic acid sequence at an appropriate location. Vector components generally include one or more of an origin of replication, one or more marker genes, an enhancer element, a promoter, and a transcription termination sequence.

The IL-1 or IL-17 antagonist may be produced recombinantly either in isolation but also by fusion to one or more other components, such as a signal sequence, an epitope or purification moiety, and a label. The IL-1 antagonist can include the pro domain of an interleukin-1 family member, e.g., which subsequently can be removed by proteolytic processing.

For bacterial expression, the IL-1 or IL-17 antagonist can be produced with or without a signal sequence. For example, it can be produced within cells so that it accumulates in inclusion bodies. It can also be secreted, e.g., by addition of a prokaryotic signal sequence, e.g., an appropriate leader sequence such as from alkaline phosphatase, penicillinase, or heat-stable enterotoxin II. Exemplary bacterial host cells for expression include any transformable E. coli K-12 strain (such as E. coli C600, ATCC 23724; E. coli HB101 NRRLB-11371, ATCC-33694; E. coli MM294 ATCC-33625; E. coli W3110 ATCC-27325), strains of B. subtilis, Pseudomonas, and other bacilli. Proteins produced in bacterial systems will typically lack glycosylation.

The IL-1 or IL-17 antagonist can be expressed in a yeast host cell, e.g., Saccharomyces cerevisiae, Schizosaccharomyces pombe, Hanseula, or Pichia pastoris. For yeast expression, the IL-1 antagonist can also be produced intracellularly or by secretion, e.g., using the yeast invertase leader or alpha factor leader (including Saccharomyces and Kluyveromyces forms), or the acid phosphatase leader, or the C. albicans glucoamylase leader (EP 362,179 published 4 Apr. 1990). In mammalian cell expression, mammalian signal sequences may be used to direct secretion of the protein, such as signal sequences from secreted polypeptides of the same or related species, as well as viral secretory leaders. Alternatively, the IL-1 antagonist can be produced with a pro domain of an interleukin-1 family member, e.g., an IL-1α or IL-1β pro domain.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria; the 2 μ plasmid origin is suitable for yeast; and various viral origins (SV40, polyoma, adenovirus, VSV or BPV) are useful for cloning vectors in mammalian cells.

Expression and cloning vectors typically contain a selection gene or marker. Typical selection genes encode proteins that (a) confer resistance to antibiotics or other toxins, e.g., ampicillin, neomycin, methotrexate, or tetracycline, (b) complement auxotrophic deficiencies (such as the URA3 marker in Saccharomyces), or (c) supply critical nutrients not available from complex media, e.g., the gene encoding D-alanine racemase for Bacilli. Various markers are also available for mammalian cells, e.g., DHFR or thymidine kinase. DHFR can be used in conjunction with a cell line (such as a CHO cell line) deficient in DHFR activity, prepared and propagated as described by Urlaub et al., Proc. Natl. Acad. Sci. USA, 77:4216 (1980).

Expression and cloning vectors usually contain a promoter operably linked to the nucleic acid sequence encoding an IL-1 or IL-17 antagonist to direct mRNA synthesis.

Exemplary promoters suitable for use with prokaryotic hosts include the β-lactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980); EP 36,776), and hybrid promoters such as the tac promoter (deBoer et al., Proc. Natl. Acad. Sci. USA, 80:21-25 (1983)). Promoters for use in bacterial systems can also contain an appropriately located Shine-Dalgarno sequence. The T7 polymerase system can also be used to drive expression of a nucleic acid coding sequence placed under control of the T7 promoter. See, e.g., the pET vectors (Invitrogen, Inc., Carlsbad Calif., USA).

Exemplary promoters for use with yeast cells include the promoters for 3-phosphoglycerate kinase, other glycolytic enzymes, and inducible promoters such as the promoter regions for alcohol dehydrogenase 2, isocytochrome C, acid phosphatase, metallothionein, and enzymes responsible for maltose and galactose utilization.

Expression of mRNA encoding a IL-1 or IL-17 antagonist from vectors in mammalian host cells can controlled, for example, by promoters obtained from the genomes of viruses such as polyoma virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters. Heterologous promoter systems can also be used, e.g., promoters responsive to tetracycline. See Urlinger, S., et al. (2000) Proc. Natl. Acad. Sci. USA 97(14):7963-7968. Transcription can also be driven by an enhancer sequence, located in cis or trans. Exemplary mammalian enhancer sequences include those for globin, elastase, albumin, α-fetoprotein, and insulin. Additional examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the coding sequence for the IL-1 or IL-17 antagonist, but is preferably located at a site 5′ from the promoter.

Expression vectors used in eukaryotic host cells (yeast, fungi, insect, plant, animal, human, or nucleated cells from other multicellular organisms) can also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the IL-1 or IL-17 antagonist. The expression vector may also include one or more intronic sequences.

An IL-1 or IL-17 antagonist can also be expressed in insect cells, e.g., Sf9 or SF21 cells, e.g., using the pFAST-BAC™0 system. Still other methods, vectors, and host cells suitable for adaptation to the synthesis of IL-1 antagonist in recombinant cells are described in Molecular Cloning: A Laboratory Manual, Third Ed., Sambrook et al. (eds.), Cold Spring Harbor Press, (2001) (ISBN: 0879695773).

Once expressed in cells, IL-1 or IL-17 antagonists can be recovered from culture medium, inclusion bodies, or cell lysates. Cells can be disrupted by various physical or chemical means, such as freeze-thaw cycling, sonication, mechanical disruption, or cell lysing agents (e.g., detergents).

IL-1 or IL-17 antagonists can be purified from other cell proteins or polypeptides that can be found in cell lysates or in the cell medium. Exemplary of purification procedures include: by fractionation on an ion-exchange column; ethanol precipitation;

reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and affinity columns. Various methods of protein purification may be employed and such methods are known in the art and described for example in Deutscher, Methods in Enzymology, 182 (1990); and Scopes, Protein Purification: Principles and Practice, Springer-Verlag, New York (2010) (ISBN: 1441928332). Purification moieties (such as epitope tags and affinity handles) can be optionally removed by proteolytic cleavage.

### Activity Assays

The ability of an IL-1 antagonist to function as antagonize an IL-1 cytokine activity can be evaluated, e.g., in a cell based assay or in vivo. It is possible to evaluate inhibition of IL-1β and/or IL-1α activity.

In one exemplary assay, the ability of an IL-1 antagonist is evaluated for its ability to inhibit IL-1β stimulated release of IL-6 from human fibroblasts. Inhibition of IL-1β-stimulated cytokine release in MRC5 cells is correlated with the agent's ability to inhibit IL-1 mediated activity in vivo. Details of the assay are described in Dinarello et al., Current Protocols in Immunology, Ch. 6.2.1-6.2.7, John Wiley and Sons Inc., 2000. Briefly, human MRC5 human fibroblasts (ATCC # CCL-171, Manassas Va., USA) are grown to confluency in multi-well plates. Cells are treated with titrated doses of the IL-1 antagonist and controls. Cells are subsequently contacted with 100 pg/ml of IL-1β in the presence of the titrated agent and/or controls. Negative control cells are not stimulated with IL-1β. The amounts of IL-6 released in each group of treated cells is measured using an IL-6 ELISA kit (e.g., BD Pharmingen (Franklin Lakes, N.J.)). Controls that can be used include buffer alone, IL-1Ra, and antibodies to IL-1R.

Efficacy of an IL-1 antagonist can also be evaluated in vivo. An exemplary assay is described in Economides et al., Nature Med., 9:47-52 (2003). Briefly, mice are injected intraperitoneally with titrated doses of the IL-1 antagonist and controls. Twenty-four hours after injection, mice are injected subcutaneously with recombinant human IL-1β at a dose of 1 μg/kg. Two hours after injection of the IL-1β (peak IL-6 response time), mice are sacrificed, and blood is collected and processed for serum. Serum IL-6 levels are assayed by ELISA. Percent inhibition can be calculated based on the ratio of IL-6 detected in experimental animal serum to IL-6 detected in controls. Another exemplary assay for an antagonist includes ability to inhibit fever mediated by IL-1.

Exemplary assays for IL-17 antagonism are described in Gerhardt et al, J. Mol. Biol. 394:905-921 (2009) and Fossiez et al. J Exp Med. 1996 Jun 1;183(6):2593-603. For example, the ability of an antagonist of IL-17A can be evaluated in an HT1080/IL-6 release assay. Cells are incubated in the presence and absence of the antagonist and recombinant IL-17A, and subsequently IL-6 in the supernatant is measured. Exemplary antagonists can reduce IL-6 production by at least 2, 5, 10, 15, or 20% relative to untreated controls. Further, the assay can be used to evaluate an IC50 of an antagonist. Exemplary antagonists can have an IC50 that is less than 100 nM, 50 nM, or 10 nM. Other exemplary assays include evaluating effects on primary chondrocytes, e.g., as described in Gerhardt et al. Still other assays include the evaluating reporter gene expression responsive to IL-17A and evaluating the ability of an antagonist to inhibit such expression. See e.g., Faour et al. J. Biol. Chem. (2003) 278, 26897-26907.

All patents, patent applications, and references are incorporated herein for all purposes. Other embodiments are within the scope of the following claims.

What is claimed is:

1. A method of administering an IL-1 or IL-17 antagonist, the method comprising topically administering an ophthalmic composition comprising an IL-1 or IL-17 antagonist to the eye of a subject one hour or less prior to sleep.

2. The method of claim 1 wherein the ophthalmic composition is in the form of an eye drop.

3. The method of claim 1 wherein the ophthalmic composition is in the form of a dissolvable implant.

4. The method of claim 1 wherein the ophthalmic composition comprises a biodegradable matrix.

5. The method of claim 4 wherein the matrix dissolves or otherwise dissembles within 6 hours.

6. A method of administering an IL-1 antagonist, the method comprising topically administering an ophthalmic composition comprising an IL-1 antagonist to the eye of a subject after an evening meal.

7. The method of claim 6 wherein the ophthalmic composition is administered at least once each day in the evening or late evening.

8. The method of claim 6 wherein the ophthalmic composition is administered at least twice daily, and wherein the final dose is administered immediately prior to sleep.

9. The method of claim 6 wherein the ophthalmic composition is administered at least twice daily, and wherein the final dose contains a higher concentration than earlier doses.

10. The method of claim 1 wherein the composition is a cotton pledget.

11. The method of claim 1 wherein the composition comprises a viscosity agent.

12. The method of claim 1 wherein the composition comprises hydroxypropyl cellulose.

13. A method of administering an IL-1 antagonist, the method comprising topically administering an ophthalmic composition comprising an IL-1 antagonist to the eye wherein the method further comprises subsequently closing the eye for at least 30 minutes and/or reducing tear drainage.

14. The method of claim 13 further comprising reducing tear drainage.

15. The method of claim 13 wherein tear drainage is reduced by insertion of a punctal plug.

16. A method of administering an IL-1 antagonist, the method comprising topically administering an IL-1 antagonist and a corticosteroid to the eye of a subject for an induction period, and administering the IL-1 antagonist in the absence of the corticosteroid during a maintenance period.

17. The method of claim 16 wherein the induction period is two weeks or less.

18. The method of claim 16 wherein the IL-1 antagonist and the corticosteroid are formulated in a single ophthalmic formulation for administration during the induction period.

19. The method of claim 16 wherein the subject is not given any corticosteroids subsequent to the induction period.

20. A method of administering an IL-1 antagonist, the method comprising topically administering an IL-1 antagonist and a corticosteroid to the eye of a subject who is a non-responder to topical cyclosporine treatment for inflammatory eye disease.


SEQUENCE LISTING (33 sequences)

SEQ ID NO: 1 - protein, 11 aa, Artificial Sequence
  Description of Artificial Sequence Synthetic peptide
RASQDISNYLS

SEQ ID NO: 2 - protein, 7 aa, Artificial Sequence
  Description of Artificial Sequence Synthetic peptide
YTSKLHS

SEQ ID NO: 3 - protein, 9 aa, Artificial Sequence
  Description of Artificial Sequence Synthetic peptide
LQGKMLPWT

SEQ ID NO: 4 - protein, 7 aa, Artificial Sequence
  Description of Artificial Sequence Synthetic peptide
TSGMGVG

SEQ ID NO: 5 - protein, 15 aa, Artificial Sequence
  Description of Artificial Sequence Synthetic peptide
HIWWDGDESYNPSLK

SEQ ID NO: 6 - protein, 10 aa, Artificial Sequence
  Description of Artificial Sequence Synthetic peptide
NRYDPPWFVD

SEQ ID NO: 7 - protein, 137 aa, Artificial Sequence
  Description of Artificial Sequence Synthetic polypeptide
MEFGLSWVFLVALLRGVQCQVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSS

SEQ ID NO: 8 - protein, 125 aa, Artificial Sequence
  Description of Artificial Sequence Synthetic polypeptide
MLPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDI

SEQ ID NO: 9 - protein, 152 aa, Artificial Sequence
  Description of Artificial Sequence Synthetic polypeptide
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 10 - protein, 14 aa, Artificial Sequence
  Description of Artificial Sequence Synthetic peptide
FTWEESNAYYWQPY

SEQ ID NO: 11 - protein, 21 aa, Artificial Sequence
  Description of Artificial Sequence Synthetic peptide
ETPFTWEESNAYYWQPYALPL

SEQ ID NO: 12 - protein, 11 aa, Artificial Sequence
  Description of Artificial Sequence Synthetic peptide
FEWTPGYWQPY

SEQ ID NO: 13 - protein, 11 aa, Artificial Sequence
  Description of Artificial Sequence Synthetic peptide
FEWTPGYWQHY

SEQ ID NO: 14 - protein, 11 aa, Artificial Sequence
  Description of Artificial Sequence Synthetic peptide
FEWTPGWYQXY

SEQ ID NO: 15 - protein, 11 aa, Artificial Sequence
  Description of Artificial Sequence Synthetic peptide
FEWTPGWYQXY

SEQ ID NO: 16 - protein, 11 aa, Artificial Sequence
  Description of Artificial Sequence Synthetic peptide
FEWTPGWYQXY

SEQ ID NO: 17 - protein, 11 aa, Artificial Sequence
  Description of Artificial Sequence Synthetic peptide
FAWTPGYWQXY

SEQ ID NO: 18 - protein, 11 aa, Artificial Sequence
  Description of Artificial Sequence Synthetic peptide
FEWAPGYWQXY

SEQ ID NO: 19 - RNA, 23 nt, Artificial Sequence
  Description of Artificial Sequence Synthetic oligonucleotide
uguaaacauccuacacucucagc

SEQ ID NO: 20 - RNA, 21 nt, Artificial Sequence
  Description of Artificial Sequence Synthetic oligonucleotide
uguaaacauccuacacucagc

SEQ ID NO: 21 - RNA, 23 nt, Artificial Sequence
  Description of Artificial Sequence Synthetic oligonucleotide
uguaaacauccucgacuggaagc

SEQ ID NO: 22 - RNA, 22 nt, Artificial Sequence
  Description of Artificial Sequence Synthetic oligonucleotide
uggcucaguucagcaggaacag

SEQ ID NO: 23 - RNA, 23 nt, Artificial Sequence
  Description of Artificial Sequence Synthetic oligonucleotide
uauggcuuuucauuccuauagug

SEQ ID NO: 24 - RNA, 20 nt, Artificial Sequence
  Description of Artificial Sequence Synthetic oligonucleotide
ccucugggcccuuccuccag

SEQ ID NO: 25 - RNA, 22 nt, Artificial Sequence
  Description of Artificial Sequence Synthetic oligonucleotide
uggacggagaacugauaagggu

SEQ ID NO: 26 - RNA, 21 nt, Artificial Sequence
  Description of Artificial Sequence Synthetic oligonucleotide
acagcaggcacagacaggcag

SEQ ID NO: 27 - RNA, 22 nt, Artificial Sequence
  Description of Artificial Sequence Synthetic oligonucleotide
gugaaauguuuaggaccacuag

SEQ ID NO: 28 - RNA, 21 nt, Artificial Sequence
  Description of Artificial Sequence Synthetic oligonucleotide
gccccugggccuauccuagaa

SEQ ID NO: 29 - RNA, 22 nt, Artificial Sequence
  Description of Artificial Sequence Synthetic oligonucleotide
uccuucauuccaccggagucug

SEQ ID NO: 30 - protein, 127 aa, Artificial Sequence [sequence omitted]

SEQ ID NO: 31 - protein, 109 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 32 - protein, 118 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDLIHGVTRNWGQGTLVTVSS

SEQ ID NO: 33 - protein, 112 aa, Artificial Sequence
  Description of Artificial Sequence Synthetic polypeptide
NFMLTQPHSVSESPGKTVTISCTRSSGSLANYYVQWYQQRPGSSPTIVIFANNQRPSGVPDRFSGSIDSSSNSASLTISGLKTEDEADYYCQTYDPYSVVFGGGTKLTVLGE